(19) United States
(12) Patent Application Publication
(10) Pub. No.: US 2025/0345580 A1
(43) Pub. Date: Nov. 13, 2025

(54) DEVICE FOR SUBDERMAL INSERTION OF SOLID MEDIA

(71) Applicant: Eastern Virginia Medical School, Norfolk, VA (US)

(72) Inventors: M. Melissa Peet, Norfolk, VA (US); Gustavo F. Doncel, Norfolk, VA (US); Jason Robinson, Norfolk, VA (US); Daniel Huang, Norfolk, VA (US); Thomas Wucherpfennig, Norfolk, VA (US); Meredith Clark, Norfolk, VA (US)

(21) Appl. No.: 19/201,170

(22) Filed: May 7, 2025

Related U.S. Application Data
(60) Provisional application No. 63/644,288, filed on May 8, 2024.
(60) Provisional application No. 63/750,062, filed on Jan. 27, 2025.

(51) Int. Cl. A61M 37/00

(52) U.S. Cl. CPC A61M 37/0069; A61M 2205/583; A61M 2210/04

(57) ABSTRACT

A device for subdermal insertion of solid media may include a front body defining a window. A device may include a rear body selectively coupled to the front body. A device may include a needle hub received within the front body and the rear body and movable between an extended position and a retracted position. A device may include a trocar coupled to the needle hub and including a payload retention feature.

### CROSS REFERENCE TO RELATED APPLICATIONS

This application claims the benefit of U.S. Provisional Patent Application No. 63/644,288 filed on May 8, 2024, and U.S. Provisional Patent Application No. 63/750,062 filed on Jan. 27, 2025, the entire contents of which are incorporated herein by reference.

### BACKGROUND

The disclosure relates to devices for subdermal insertion of solid media including trocar needles. More specifically, the disclosure relates to trocar needles including media retention features and devices for subdermal insertion of solid media with improved usability features.

### SUMMARY

In some aspects, the disclosure relates to a device for subdermal insertion of solid media, the device including: a front body defining a window; a rear body selectively coupled to the front body; a needle hub received within the front body and the rear body and movable between an extended position and a retracted position; and a trocar coupled to the needle hub and including a payload retention feature.

In some aspects, the disclosure relates to a device, wherein the rear body includes an indicator window, and wherein the needle hub includes a hub indicator tab that is viewable through the indicator window when the needle hub is arranged in the retracted position.

In some aspects, the disclosure relates to a device, further including a plunger positioned within the rear body and including a plunger indicator tab viewable through the indicator window when the needle hub is arranged in the extended position.

In some aspects, the disclosure relates to a device, wherein the plunger includes a rod sized to be received within the trocar and configured to eject a payload from the trocar.

In some aspects, the disclosure relates to a device, wherein the needle hub includes a hub button configured to be engaged by a user to force the needle hub from the extended position to the retracted position.

In some aspects, the disclosure relates to a device, wherein the trocar is a tab retainer trocar including a payload retainer tab.

In some aspects, the disclosure relates to a device, wherein the trocar is a sleeve tip retainer trocar including a polymer sleeve.

In some aspects, the disclosure relates to a device, wherein the trocar is a foil retainer trocar including a flat shaped foil or a v-shaped foil.

In some aspects, the disclosure relates to a device, wherein the trocar is a sleeve slot retainer trocar that defines a slot and includes a polymer sleeve covering the slot.

In some aspects, the disclosure relates to a device, wherein the trocar is a plug retainer trocar including a plug.

In some aspects, the disclosure relates to a device, wherein the rear body includes a rod sized to be received within the trocar and configured to eject a payload from the trocar.

In some aspects, the disclosure relates to a device, wherein the needle hub includes a hub button defining a pulling surface at a distal end of the hub button.

In some aspects, the disclosure relates to a device, wherein the pulling surface intersects a proximal window plane intersecting a proximal end of the window.

In some aspects, the disclosure relates to a device, wherein the window defines a window width of 14 mm or less.

In some aspects, the disclosure relates to a device, wherein the window is defined by a hoop having a width wider than a hand grip.

In some aspects, the disclosure relates to a device, wherein the rear body defines a swept tail configured to be pushed by a user.

In some aspects, the disclosure relates to a device, wherein the front body defines a textured grip surface.

In some aspects, the disclosure relates to a device for subdermal insertion of solid media, the device including: a body includes a hand grip and a loop defining a window, the loop defining a width wider than the hand grip; a needle hub slidably received within the body movable between an extended position and a retracted position; and a trocar coupled to the needle hub and including a payload retention feature.

In some aspects, the disclosure relates to a device, wherein the trocar defines a cutting tip, and wherein the cutting tip is visible through the window when the needle hub is arranged in the extended position.

In some aspects, the disclosure relates to a device for subdermal insertion of solid media, the device including: a front body includes a hand grip and a loop defining a window and a proximal window plane intersecting a proximal end of the window, wherein the window defines a window width of 14 mm or less; a rear body selectively coupled to the front body, the rear body defining a swept tail configured to be pushed by a user; a needle hub received within the front body and the rear body and movable between an extended position and a retracted position, the needle hub including a hub button defining a pulling surface at a distal end of the hub button, wherein the pulling surface intersects the proximal window plane; and a trocar coupled to the needle hub and including a payload retention feature. This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF DRAWINGS

Example features and implementations are disclosed in the accompanying drawings. However, the present disclosure is not limited to the precise arrangements and instrumentalities shown.

FIG. 1 is a perspective view of a device for subdermal insertion of solid media, according to one implementation.

FIG. 2 is a detail view of a portion of the device for subdermal insertion of solid media of FIG. 1.

FIG. 3 is an exploded view of the device for subdermal insertion of solid media of FIG. 1.

FIG. 4 is a perspective view of the device for subdermal insertion of solid media of FIG. 1 in an extended position, according to some implementations.

FIG. 5 is a perspective view of the device for subdermal insertion of solid media of FIG. 1 in an intermediate position, according to some implementations.

FIG. 6 is a perspective view of the device for subdermal insertion of solid media of FIG. 1 in a retracted position, according to some implementations.

FIG. 7 is a perspective view of the device for subdermal insertion of solid media of FIG. 1 arranged in packaging, according to some implementations.

FIG. 8 is a perspective view of a tab retainer trocar that can be used with the device for subdermal insertion of solid media of FIG. 1, according to some implementations.

FIG. 9 is another perspective view of the tab retainer trocar of FIG. 8.

FIG. 10 is a perspective view of another tab retainer trocar that can be used with the device for subdermal insertion of solid media of FIG. 1, according to some implementations.

FIG. 11 is a perspective view of a sleeve tip retainer trocar that can be used with the device for subdermal insertion of solid media of FIG. 1, according to some implementations.

FIG. 12 is a perspective view of another sleeve tip retainer trocar that can be used with the device for subdermal insertion of solid media of FIG. 1, according to some implementations.

FIG. 13 is a schematic view of a cross section of a foil retainer trocar that can be used with the device for subdermal insertion of solid media of FIG. 1, according to some implementations.

FIG. 14 is a schematic view of a cross section of another foil retainer trocar that can be used with the device for subdermal insertion of solid media of FIG. 1, according to some implementations

FIG. 15 is a perspective view of a sleeve slot retainer trocar that can be used with the device for subdermal insertion of solid media of FIG. 1, according to some implementations

FIG. 16 is another perspective view of the sleeve slot retainer of FIG. 15.

FIG. 17 is a schematic view of a plug retainer trocar that can be used with the device for subdermal insertion of solid media of FIG. 1, according to some implementations.

FIG. 18 is a perspective view of another device for subdermal insertion of solid media, according to one implementation.

FIG. 19 is a side view of the device for subdermal insertion of solid media of FIG. 18, according to some implementations.

FIG. 20 is a top view of the device for subdermal insertion of solid media of FIG. 18 in an extended position, according to some implementations.

FIG. 21 is a top view of the device for subdermal insertion of solid media of FIG. 18 in a retracted position, according to some implementations.

FIG. 22 is an exploded view of the device for subdermal insertion of solid media of FIG. 18, according to some implementations.

FIG. 23 is a perspective view of another device for subdermal insertion of solid media, according to one implementation.

FIG. 24 is a perspective view of another device for subdermal insertion of solid media, according to one implementation.

FIG. 25 is a perspective view of another device for subdermal insertion of solid media, according to one implementation.

FIG. 26 is a perspective view of another device for subdermal insertion of solid media, according to one implementation.

FIGS. 27-29 are perspective views showing components of the device for subdermal insertion of solid media of FIG. 18 moving from the extended position to the retracted position, according to some implementations.

FIGS. 30-32 are perspective views of a trocar of the device for subdermal insertion of solid media of FIG. 18 being inserted, according to some implementations.

### DETAILED DESCRIPTION

The Following below are more detailed descriptions of concepts related to, and implementations of, methods, apparatuses, and systems for the subdermal insertion of solid media. The figures illustrate exemplary implementations in detail and the present disclosure is not limited to the details or methodology set forth in the description or illustrated in the figures. The terminology used herein is for the purpose of description only and should not be regarded as limiting.

Referring to the figures generally, the various implementations disclosed herein relate to systems, apparatuses, and methods for subdermally inserting a payload of solid media in the form of medicated pellets or other form factors (e.g., for providing birth control, the treatment and/or prevention of HIV, etc.).

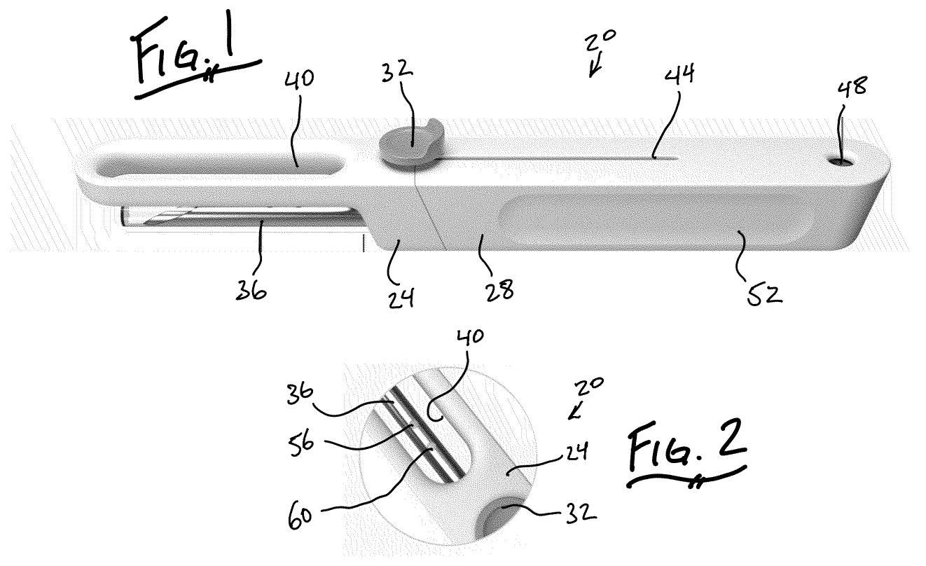

As shown in FIG. 1, a device for subdermal insertion of solid media in the form of an inserter 20 including a first or front body 24 (e.g., a loop window portion), a second or rear body 28 (e.g., a handle), a needle hub 32, and a trocar 36. In some implementations, the inserter 20 is formed with a single body, or more than two bodies. The front body 24 includes a depth-guide or window 40 defined by a hoop 42 providing a clear view of the trocar 36 during insertion. The rear body 28 defines a slide slot 44 constraining movement of the needle hub 32 along a longitudinal axis between an extended position (shown in FIG. 1) and a retracted position (see FIG. 6). The rear body 28 also includes an indicator window 48 and a hand grip 52.

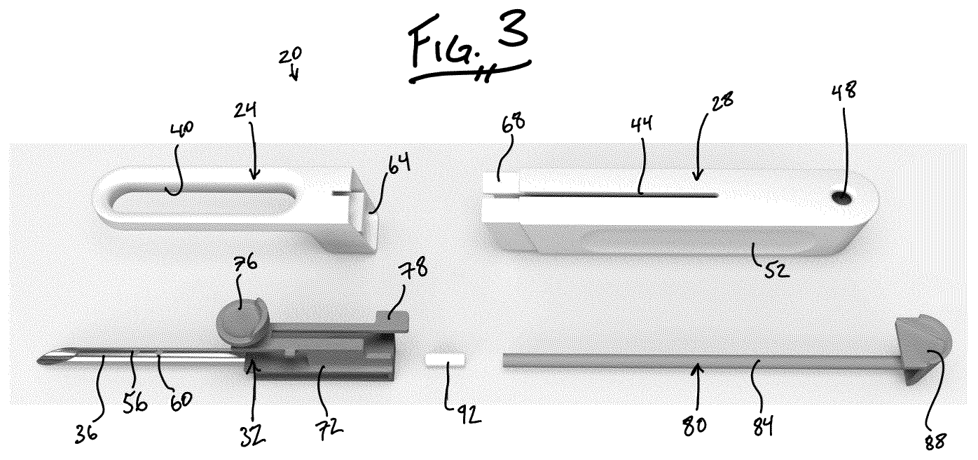

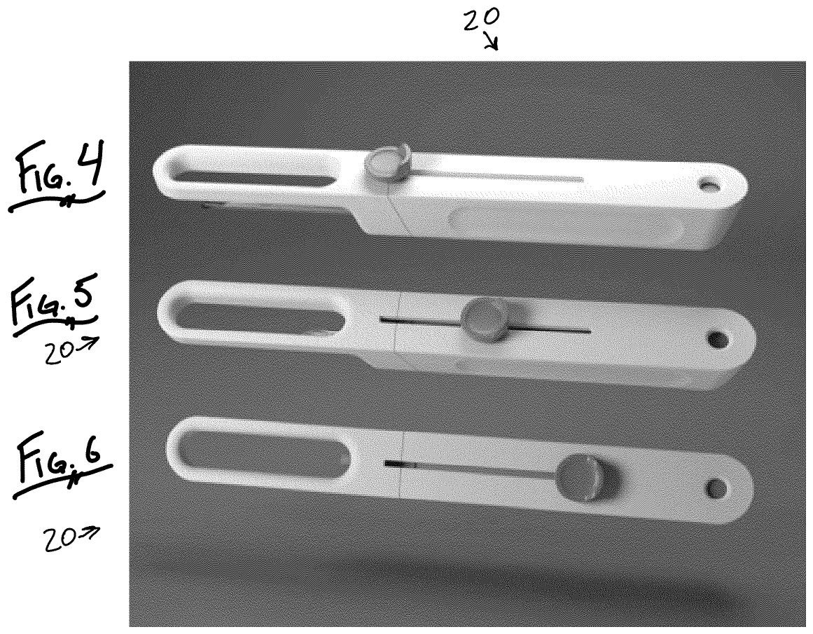

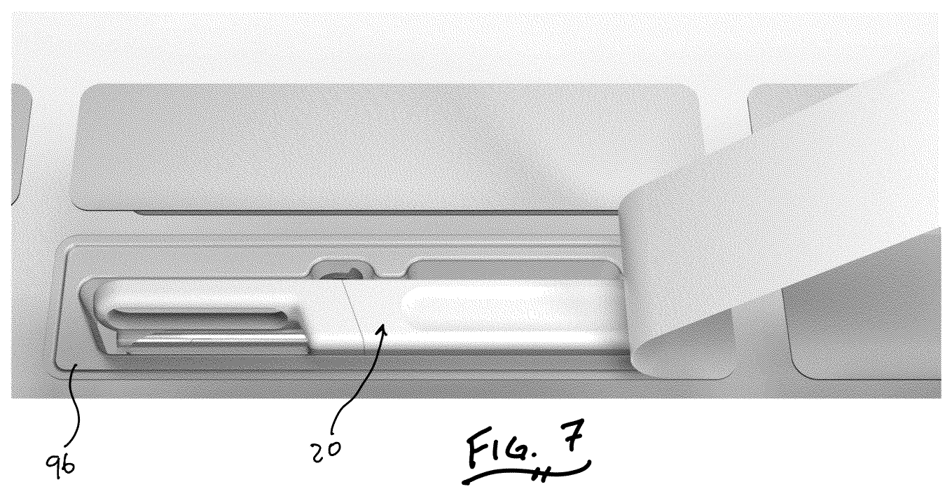

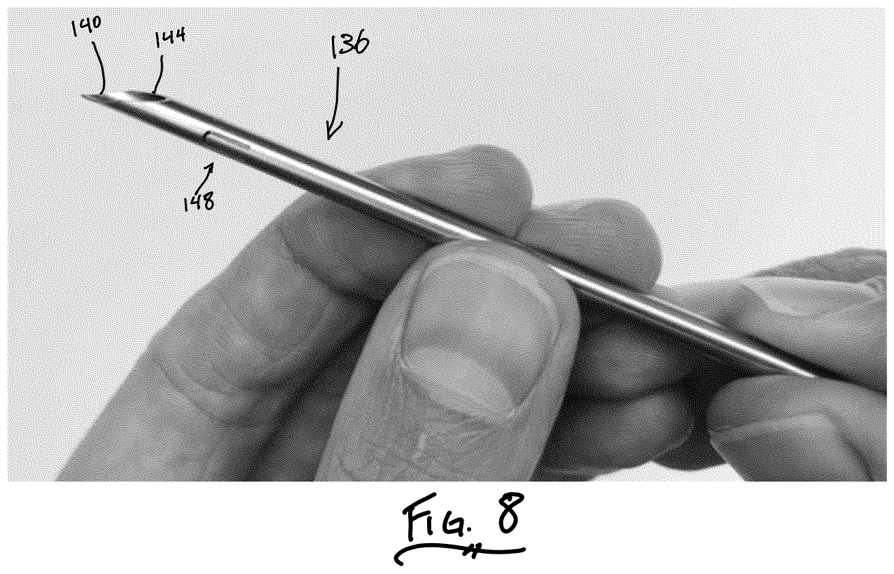

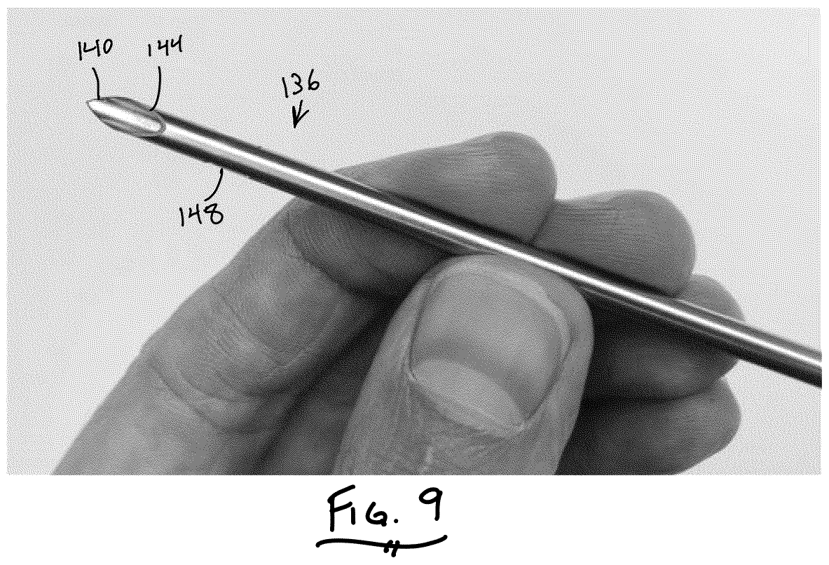

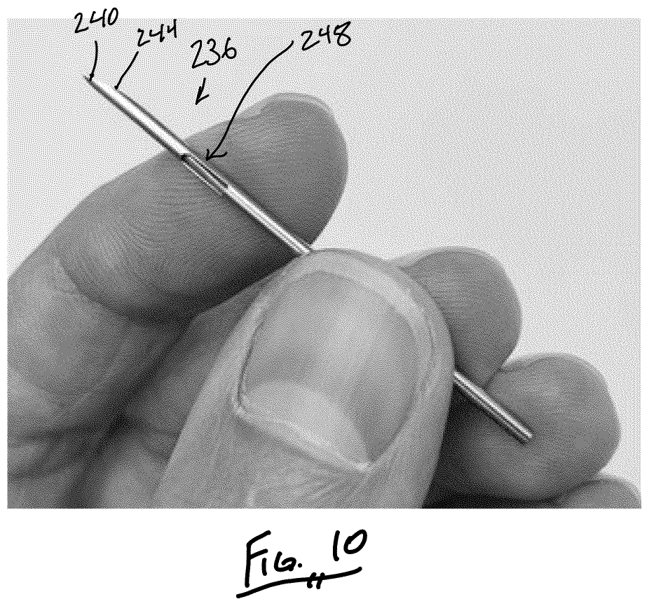

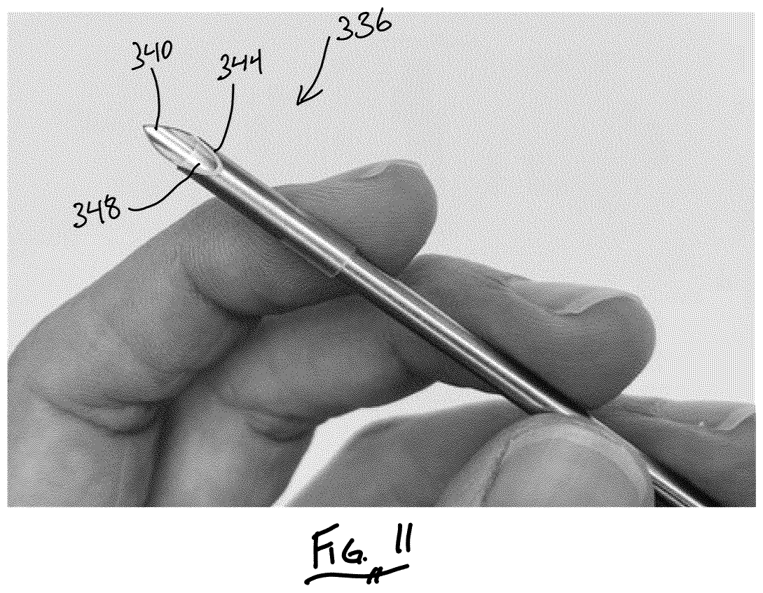

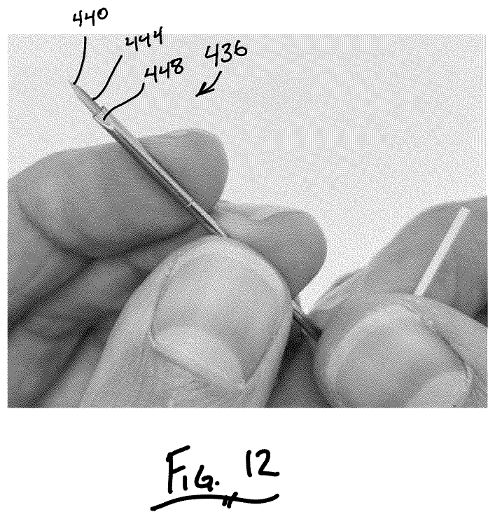

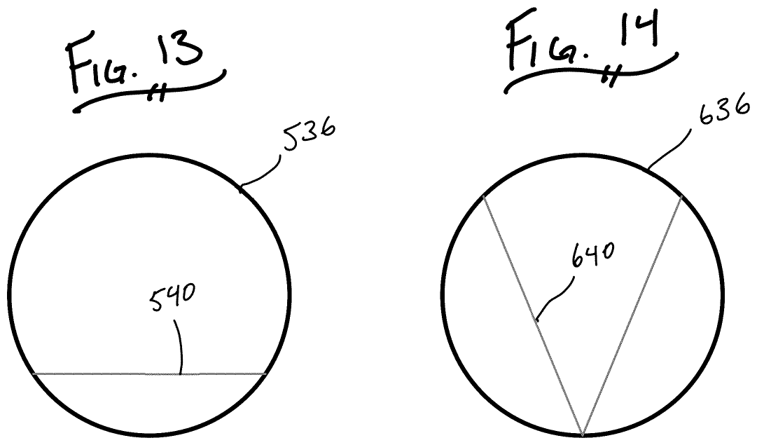

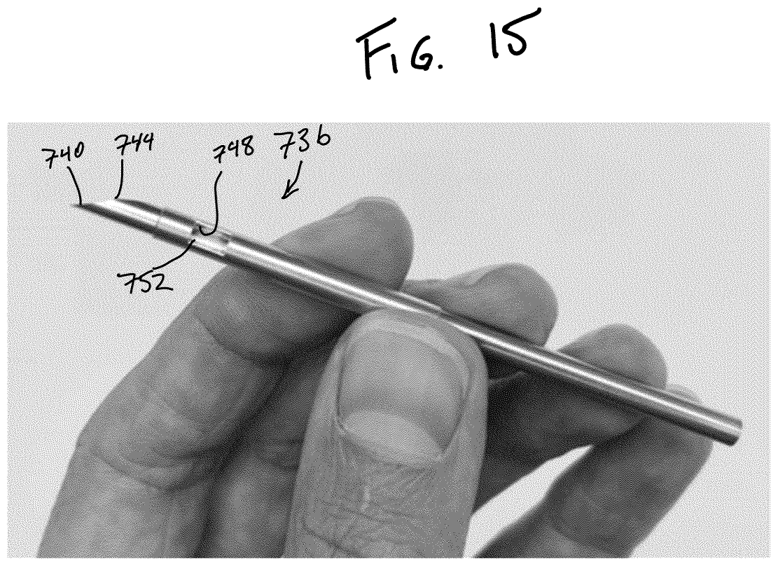

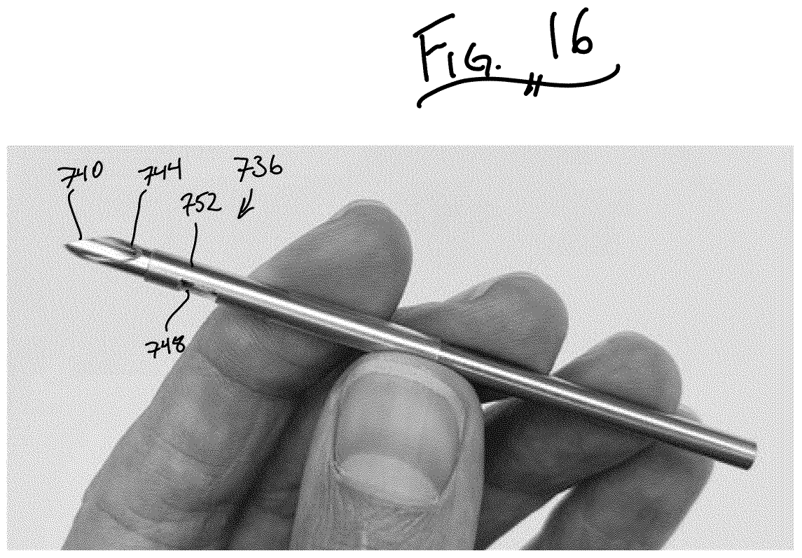

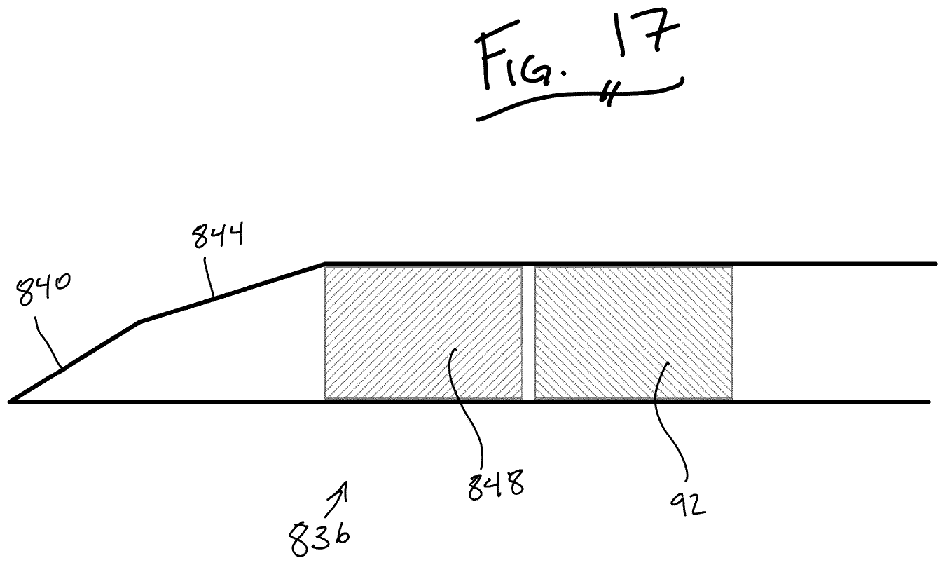

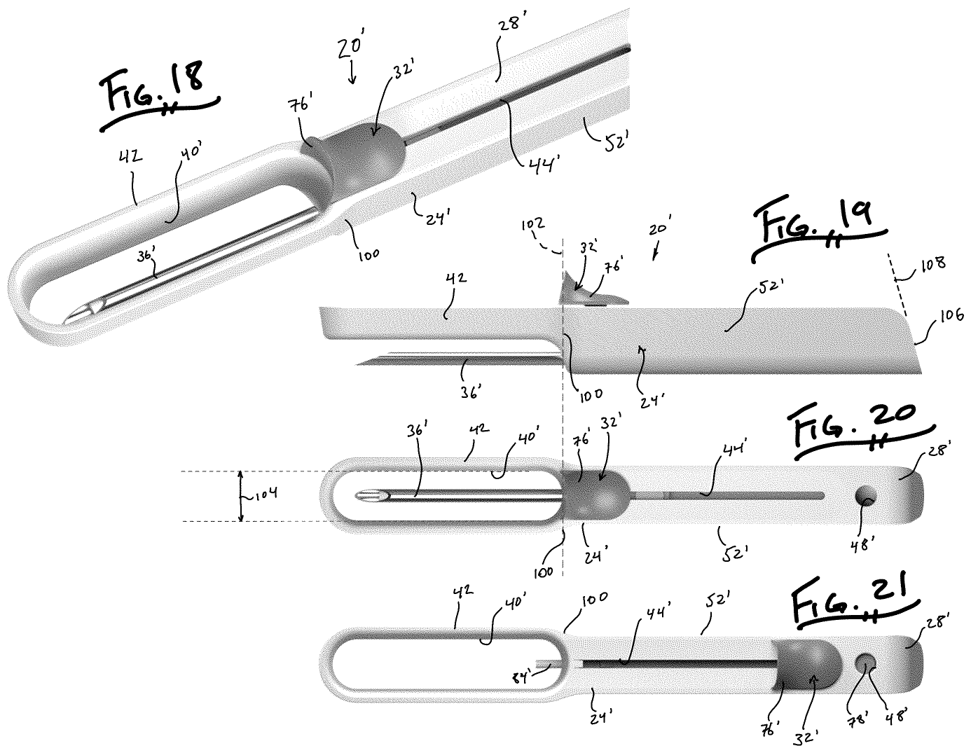

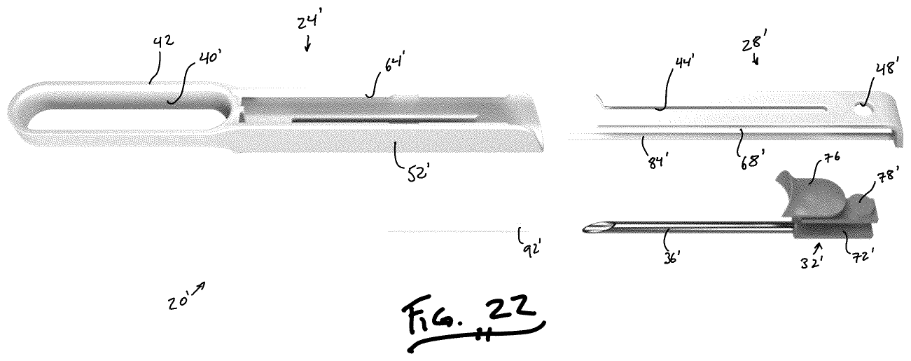

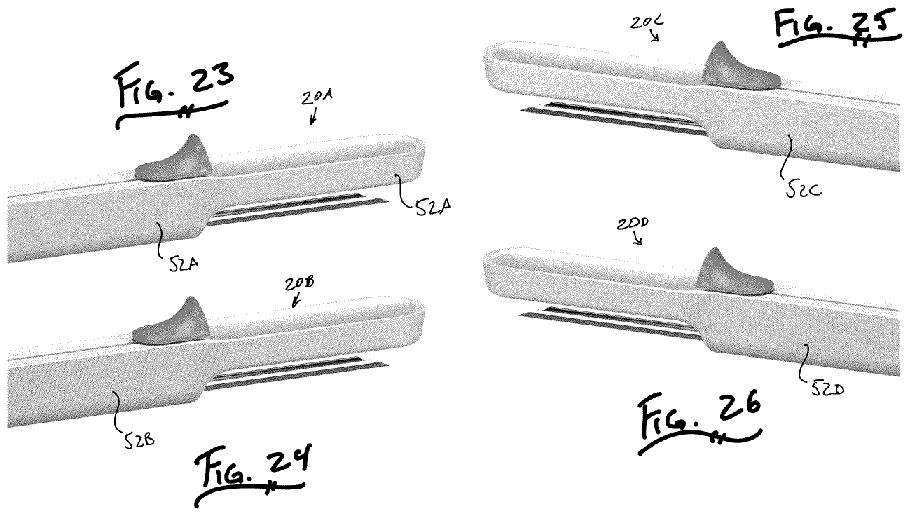

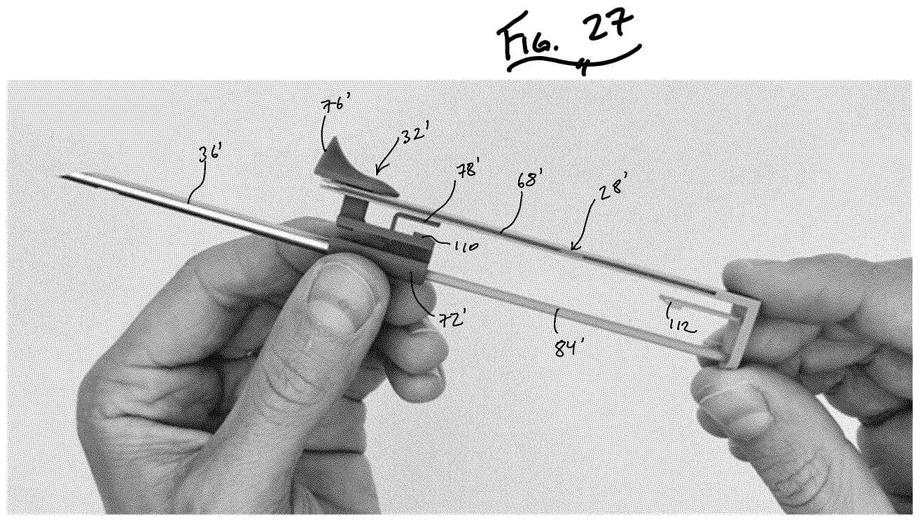

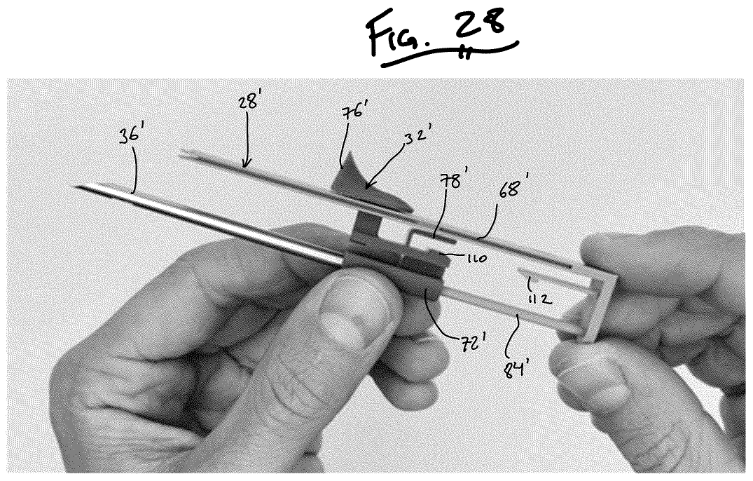

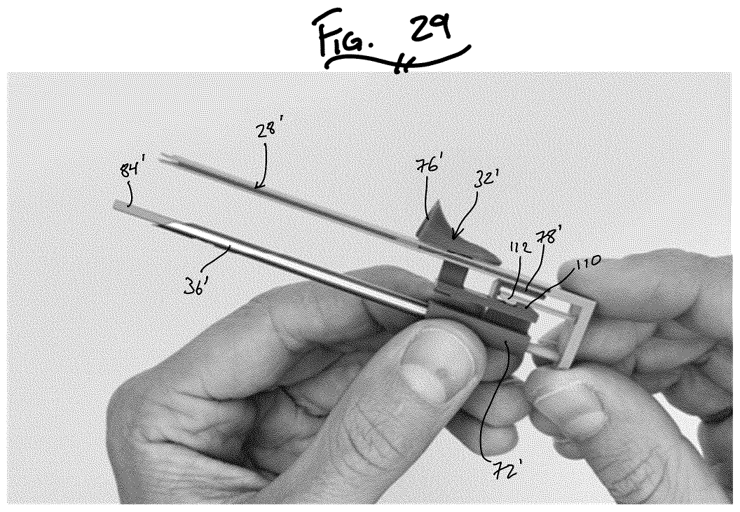

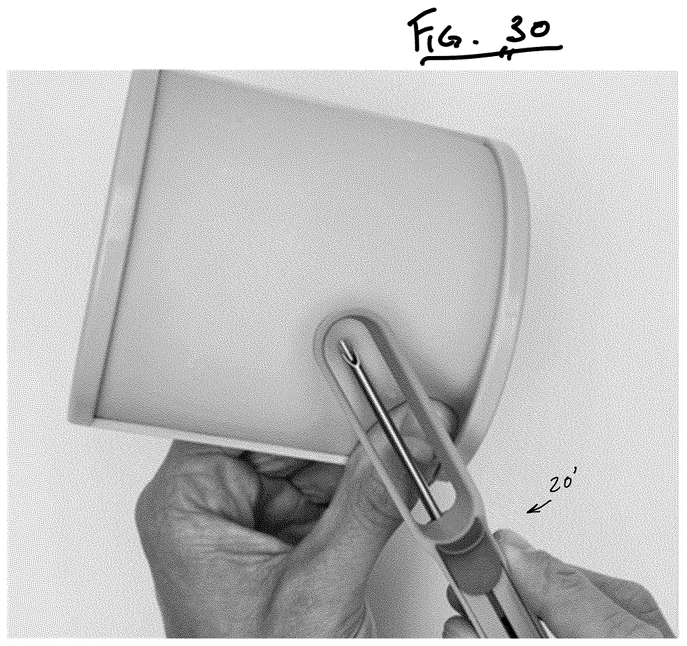

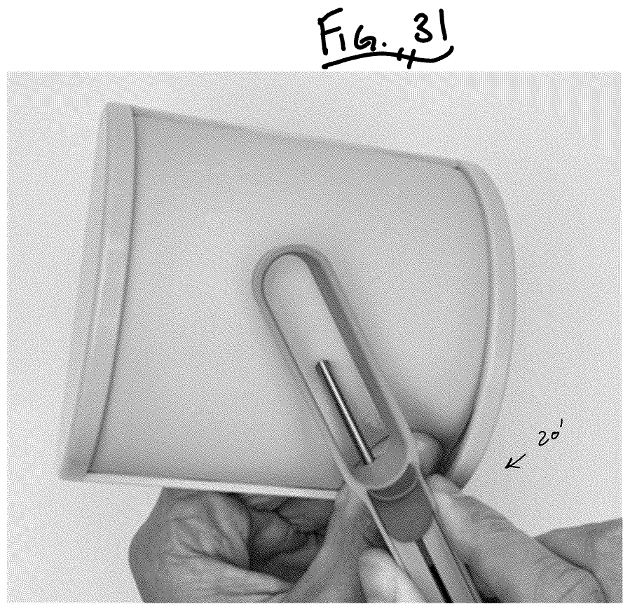

As shown in FIG. 2, the trocar 36 includes a distal loading indicator window 56 positioned adjacent a distal tip of the trocar 36 and a proximal loading indicator window 60 spaced from the distal loading indicator window 56 away from the distal tip of the trocar 36 parallel to the longitudinal axis. The distal loading indicator window 56 and the proximal loading indicator window 60 provide a user with a clear view of a payload/solid media in the form of a pellet 92 loaded into the trocar 36 (see FIG. 3). In some implementations, the pellet 92 is a different type of insert, payload, implant, rod-like implant, solid media, etc. is used with the inserter 20. The form factor of the pellet 92 depicted herein is non-limiting to the features of the inserter 20 described herein.

In some implementations, only one indicator window is included. In some implementations, more than two indicator windows are included. In some implementations, the indicator windows are eliminated. The distal loading indicator window 56 and the proximal loading indicator window 60 are arranged on a top area of the trocar 36 so they are clearly visible through the window 40 of the front body 24 during use.

As shown in FIG. 3, the front body 24 defines a front body mating feature 64 sized to receive a portion of the needle hub 32. The rear body 28 defines a rear body mating feature 68 sized to engage the front body mating feature 64. The front body 24 and the rear body 28 are selectively engageable with one another for loading pellets 92 into the trocar 36.

The needle hub 32 includes a hub body 72 sized to hold the trocar 36 and including a hub aperture matched to the size of the trocar 36. A hub button 76 is structured to extend through the slide slot 44 and be engageable by the user to actuate the inserter 20 between the extended position and the retracted position. In some implementations, the hub button 76 includes a locking feature that inhibits the trocar 36 and needle hub 32 from retracting while the trocar 36 is being inserted. A hub indicator tab 78 is spaced from the hub button 76 and positioned to interact with the indicator window 48 of the rear body 28 during use (described in more detail below).

A plunger 80 includes a rod 84 sized to be received within the trocar 36 and extend through the hub aperture. A plunger indicator tab 88 shaped so to be selectively viewable through the indicator window 48 of the rear body 28 during use (described in more detail below).

In operation, as shown in FIG. 4, the inserter 20 is arranged in the extended position with the trocar 36 fully extended and ready for insertion. The pellet 92 is loaded into the trocar 36 and visible through at least one of the distal loading indicator window 56 and/or the proximal loading indicator window 60 so the user can easily confirm the presence of the pellet 92. The plunger indicator tab 88 is clearly visible through the indicator window 48 of the rear body 28 confirming the user that the inserter 20 is arranged in the extended position. In some implementations the plunger indicator tab 88 is green in color and indicates a ready state of the inserter 20.

As shown in FIG. 5, once the trocar 36 is inserted and in the desired position, the hub button 76 of the needle hub 32 is moved parallel to the longitudinal axis rearward thereby retracting the trocar 36 into the front body 24 and the rear body 28. During retraction, the rod 84 of the plunger 80 holds the pellet 92 in place and inhibits the pellet 92 from retracting along with the trocar 36. As the needle hub 32 and the trocar 36 are retracted, the pellet 92 is ejected from the inserter 20.

As shown in FIG. 6, the inserter 20 is arranged in the retracted position with the needle hub 32 and the trocar 36 fully retracted into the front body 24 and the rear body 28 so that the pellet 92 is fully ejected and implanted subdermally. The hub indicator tab 78 is visible through the indicator window 48 of the rear body 28 indicating that the inserter 20 is used.

In some implementations, the pellet 92 is preloaded in the trocar 36. In some implementations, the trocar 36 is loaded with one or more pellets 92 by the user.

As shown in FIG. 7, in some implementations, the inserter 20 is provided in a sterile package 96 including a preloaded pellet 92 or multiple pellets 92. This allows the user to simply open the sterile package 96 and use the inserter 20 without multiple cleaning and sterilization steps. The sterile package 96 is designed to be compact and includes stacking features such that one sterile package 96 nests or partially nests with an adjacent sterile package 96.

As shown in FIGS. 8 and 9, a tab retainer trocar 136 can be used with the inserter 20. In some implementations, the tab retainer trocar 136 is a double bevel trocar and includes a cutting tip 140 and a secondary bevel 144 defining a rearward extent in a longitudinal direction away from the cutting tip 140. A pellet retainer tab 148 is formed in a sidewall of the tab retainer trocar 136 and defines a free end arranged closer to the cutting tip 140 than a fixed end. In some implementations, the pellet retainer tab 148 is formed via laser cutting. The pellet retainer tab 148 is deflected inward of the sidewall of the tab retainer trocar 136 to interfere with the pellet 92 and inhibit the pellet 92 from exiting the tab retainer trocar 136 before being forcibly ejected by the rod 84 of the plunger 80. In some implementations, the pellet retainer tab 148 includes a dimple or a scoop shape that provides a smooth surface interaction with the pellet 92 thereby inhibiting damage to the pellet 92 during ejection. In some implementations, the free end of the pellet retainer tab 148 is spaced apart from the rearward extend of the secondary bevel 144. In some implementations, the free end of the pellet retainer tab 148 is aligned with the rearward extend of the secondary bevel 144 in a plane perpendicular to the longitudinal axis. In some implementations, the free end of the pellet retainer tab 148 is closer to the cutting tip 140 than the rearward extend of the secondary bevel 144. The pellet retainer tab 148 is arranged in the sidewall of the tab retainer trocar 136 so that the distal loading indicator window 56 and the proximal loading indicator window 60 are still visible during use. In some implementations, one pellet retainer tab 148 is included (as shown in FIGS. 8 and 9). In some implementations, two pellet retainer tabs 148 are included on opposing sidewalls of the tab retainer trocar 136. In some implementations, two or more pellet retainer tabs 148 are arranged in a spaced apart array parallel to the longitudinal axis. Multiple axially spaced pellet retainer tabs 148 may be advantageous when used with multiple pellets 92. In some implementations, the tab retainer trocar 136 is a 9 gauge trocar. Having a second tab at the back end of the needle could prevent pellets from falling out the back after the have been loaded but before the plunger has been inserted. While the illustrated tab retainer trocar 136 is shown as a 9 gauge, trocar, the trocar disclosed herein can be any gauge, as desired. For example, the retainer features discussed herein can be provided on a trocar of 9 gauge to 16 gauge.

As shown in FIG. 10, another tab retainer trocar 248 is similar to the tab retainer trocar 136 discussed above and is numbers with like reference numbers in the 200 series. In some implementations, the tab retainer trocar 248 is a 16 gauge trocar.

As shown in FIG. 11, a sleeve tip retainer trocar 336 can be used with the inserter 20. In some implementations, the sleeve tip retainer trocar 336 is a double bevel trocar and includes a cutting tip 340 and a secondary bevel 344 defining a rearward extent in a longitudinal direction away from the cutting tip 340. A polymer sleeve 348 surrounds the sleeve tip retainer trocar 336 so that a forward portion of the polymer sleeve 348 extends over the secondary bevel 344 and extends into the trocar aperture to inhibit the pellet 92 from exiting the polymer sleeve 348 until it is forcibly ejected by the rod 84 of the plunger 80. In some implementations, the polymer sleeve 348 is shrink wrapped unto the sleeve tip retainer trocar 336. In some implementations, the polymer sleeve 348 is co-molded or overmolded. In some implementations, the polymer sleeve 348 extends along a length of the sleeve tip retainer trocar 336 so that a rearward extent of the polymer sleeve 348 does not enter the patient during use and therefore inhibits snagging or discomfort during ejection of the pellet 92 as the inserter 20 is moved toward the retracted position. In some implementations, the sleeve tip retainer trocar 336 is a 9 gauge trocar. In some implementations, the polymer sleeve 348 is clear, allowing pellet 92 to be confidently seen loaded in the sleeve tip retainer trocar 336.

As shown in FIG. 12, another sleeve tip retainer trocar 436 is similar to the sleeve tip retainer trocar 336 discussed above and is numbers with like reference numbers in the 400 series. In some implementations, the sleeve tip retainer trocar 436 is a 14 gauge trocar.

As shown in FIG. 13, a foil retainer trocar 536 can be used with the inserter 20. The foil retainer trocar 536 includes a flat shaped foil 540 arranged within the trocar aperture and extending along a length of the foil retainer trocar 536. In some implementations, the flat shaped foil 540 is secured at a proximal end of the foil retainer trocar 536 opposite a cutting tip. For example, the flat shaped foil 540 can be secured in place by the hub body 72. The flat shaped foil 540 is structured to deform (e.g., elastic or plastic deformation) as the pellet 92 is engaged with the flat shaped foil 540 thereby holding the pellet 92 in place and inhibiting the pellet 92 from exiting the foil retainer trocar 536 until it is forcibly ejected by the rod 84 of the plunger 80.

As shown in FIG. 14, another foil retainer trocar 636 can be used with the inserter 20 and is functionally similar to the foil retainer trocar 536 discussed above. The foil retainer trocar 636 includes a v-shaped foil 640. In some implementations, other foil shapes may be implemented to inhibit undesirable exiting of the pellet 92 from the foil retainer trocar 636.

As shown in FIGS. 15 and 16, a sleeve slot retainer trocar 736 can be used with the inserter 20. In some implementations, the sleeve slot retainer trocar 736 is a double bevel trocar and includes a cutting tip 740 and a secondary bevel 744 defining a rearward extent in a longitudinal direction away from the cutting tip 740. The sleeve slot retainer trocar 736 defines a slot 748 formed in a sidewall of the trocar. In some implementations, the slot 748 is positioned rearward or spaced apart from the rearward extent of the secondary bevel 744.

A polymer sleeve 752 surrounds the sleeve slot retainer trocar 736 so that the portion of the polymer sleeve 752 covering the slot 748 extends into the trocar aperture to inhibit the pellet 92 from exiting the sleeve slot retainer trocar 736 until it is forcibly ejected by the rod 84 of the plunger 80. In some implementations, the polymer sleeve 752 is shrink wrapped unto the sleeve slot retainer trocar 736. In some implementations, the polymer sleeve 752 is co-molded or overmolded. In some implementations, the polymer sleeve 752 extends along a length of the sleeve slot retainer trocar 736 so that a rearward extent of the polymer sleeve 752 does not enter the patient during use and therefore inhibits snagging or discomfort during ejection of the pellet 92 as the inserter 20 is moved toward the retracted position. In some implementations, the sleeve slot retainer trocar 736 is a 9 gauge trocar. In some implementations, the polymer sleeve 752 is clear, allowing pellet 92 to be confidently seen loaded in the sleeve slot retainer trocar 736.

As shown in FIG. 17, a plug retainer trocar 836 can be used with the inserter 20. In some implementations, the plug retainer trocar 836 is a double bevel trocar and includes a cutting tip 840 and a secondary bevel 844 defining a rearward extent in a longitudinal direction away from the cutting tip 840. A plug 848 is arranged within the trocar aperture and frictionally engaged therein to inhibit removal of the pellet 92 until the inserter 20 is moved toward the retracted position. In some implementations, the plug 848 is positioned rearward of the rearward extent of the secondary bevel 844. In some implementations, the plug 848 is positioned even with the rearward extent of the secondary bevel 844. In some implementations, the plug 848 is shaped to correspond with the secondary bevel 844 and the forward edge of the plug 848 is forward of the rearward extent of the secondary bevel 844 toward the cutting tip 840. The plug retainer trocar 836 is formed of a biocompatible material and is implanted with the pellet 92 during use. In some implementations, the plug 848 is an inert biocompatible material that rapidly dissolves in the body of the patient but is shelf stable in storage.

As shown in FIG. 18, another inserter 20′ is similar to the inserter 20 discussed above and like components are numbered with reference numerals in the prime series. The hoop 42′ of the inserter 20′ is wider than the hand grip 52′ and defines a hoop shoulder 100 between the hoop 42′ and the hand grip 52′. In some implementations, the hoop shoulder 100 is intersected by a proximal window plane 102 (see FIGS. 19 and 20) extending through a proximal end of the window 40′. The hoop shoulder 100 provides a flared shape that a user can use for leverage while inserting the trocar 36′. Inserting the trocar 36′ into the skin can require a good degree of force. The hoop shoulder 100 creates an affordance for the user to push against with their fingers while inserting the trocar 36′ into the skin.

As shown in FIGS. 20 and 21, the window 40′ defines a window width 104 of less that 14 mm. In some implementations, the window width 104 is 13 mm to 15 mm. The hoop 42′ provide a wider viewing angle to the trocar 36′. The wider hoop 42′ also enables better payload placement and pre-visualization during the procedure. The window 40′ allows for a clear line of sight to the full length of the trocar 36′ from tip to base. This allows the clinician to confirm that the trocar 36′ is fully inserted into the tissue before the hub button 76′ is engaged to retract the needle hub 32′ and the trocar 36′, thus inhibiting the pellet 92′ from a being partially inserted under the skin.

In some implementations, walls of the hoop 42′ are taller than the wall of the depth-guide or window 40 discussed above and treated with a grip texture to allow a secure and discernible touchpoint to anchor the device during retraction. Gripping the inserter 20′ with a second hand before pulling back the hub button 76′ inhibits the inserter 20′ and trocar 36′ position from accidentally shifting backward and risking a partial insertion of the pellet 92′. The window width 104 is large enough to allow a finger to comfortably access the pulling surface of the hub button 76′ and allow it to be pulled up and backwards. This affordance makes the unique pull up release mechanism more intuitive and makes the device design more compact and elegant.

The hoop 42′ and window 40′ of the inserter 20′ provide improved insertion visibility, needle depth visibility, provide an improved grip surface for pushing during insertion, provide improved access to the hub button 76′, and improves the ability for the user to utilize a two handed retraction technique.

The hub button 76′ of the needle hub 32′ is shaped so that a distal end of the hub button 76′ provides a pulling surface. In some implementations, the pulling surface of the hub button 76′ is arranged at an acute angle relative to the proximal window plane 102 (see FIG. 19) and intersects the proximal window plane 102 when the needle hub 32′ is in the extended position. Positioning the pulling surface of the hub button 76′ at the distal end of the hub button 76′ provides a large surface that gives the user a good purchase on the needle hub 32′ giving them better control over the release, the trocar retraction speed, and inhibits fingers from slipping off during the procedure. Additionally, the pulling surface of the hub button 76′ can be textured to improve grip and control. In some implementations, a concave pulling surface indicates to the user that the hub button 76′ should be pulled up rather than pushed down. Having a trocar 36′ release with a pull up action rather than a push down action reduces the risk that users will accidentally release the needle prematurely during needle insertion.

As shown in FIG. 19, a proximal tail 106 of the inserter 20′ is swept or angled relative to a top surface of the inserter 20.′ In some implementations, the proximal tail 106 is arranged in a swept tail plane 108 that is angled relative to the top surface of the inserter 20′. The swept tail plane 108 is arranged at an oblique angle relative to the top surface to provide an ergonomic surface for the clinician to push against with their palm during needle insertion.

As shown in FIG. 22, the front body 24′ of the inserter 20′ is formed to include the hand grip 52′ that runs along the sides of the inserter 20′ and the front body mating feature 64′ is formed adjacent a top surface of the front body 24′. The rear body 28′ is formed to define the proximal tail 106 and is formed as a single piece with the rod 84′. The rear body mating feature 68′ includes tabs shaped to engage the front body mating feature 64′ of the front body 24′. To assemble the rear body 28′ to the front body 24′ the rear body mating feature 68′ are slid into the front body mating feature 64′. In some implementations, the front body mating feature 64′ and the rear body mating feature 68′ lock together. In some implementations, the front body mating feature 64′ are fastened, adhered, or otherwise fixed to the rear body mating feature 68′. This assembly architecture reduces the number of components needed to make the inserter 20′ which in turn simplifies the assembly process, reduces costs, and makes the inserter 20′ more secure, tamper proof, and robust.

As shown in FIG. 23, an inserter 20A is similar to the inserter 20′ discussed above and includes a hand grip 52A defining two areas of localized textured strips.

As shown in FIG. 24, an inserter 20B is similar to the inserter 20′ discussed above and includes a hand grip 52B defining a full length of slanted textured strips extending from the main body of the front body 24′ to the hoop 42′.

As shown in FIG. 25, an inserter 20C is similar to the inserter 20′ discussed above and includes a hand grip 52C defining a textured (marbled, bumpy, shark skin, etc.) surface finish on the exterior of the front body 24′ and/or the hoop 42′.

As shown in FIG. 26, an inserter 20D is similar to the inserter 20′ discussed above and includes a hand grip 52D defining a full length of vertical textured strips extending from the main body of the front body 24′ to the hoop 42′.

As shown in FIGS. 27-29, the needle hub 32′ can be actuated from an extended position to a retracted position relative to the rear body 28′. In some implementations, the needle hub 32′ includes a retraction lock feature in the form of a post 110 and the rear body 28′ includes a retraction lock feature in the form of a latch 112. As shown clearly in FIG. 29, when the needle hub 32′ is arranged in the retracted position, the post 110 engages the latch 112 and the needle hub 32′ is inhibited from returning to the extended position. In some implementations, a distal end of the plunger 84′ is visible through the window 40′ when the device 20′ is arranged in the retracted position.

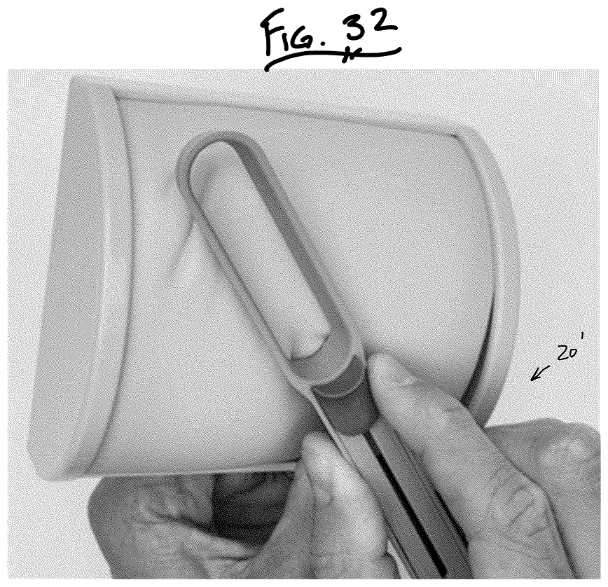

As shown in FIGS. 30-32, the wider window 40′ of the inserter 20′ provides a clear view of the trocar 36′ during an insertion process. As is most clearly shown in FIG. 30, the entire trocar 36′ is clearly visible via the window 40′. The trocar 36′ is clearly visible via the window 40′ until it is completely inserted subdermally as shown in FIG. 32.

For purposes of this description, certain advantages and novel features of the aspects and configurations of this disclosure are described herein. The described methods, systems, and apparatus should not be construed as limiting in any way. Instead, the present disclosure is directed toward all novel and nonobvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

Features disclosed in this specification (including any accompanying claims, abstract, and drawings), and/or all of the steps of any method or process so disclosed, may be combined in any combination, except combinations where at least some of such features and/or steps are mutually exclusive. The claimed features extend to any novel one, or any novel combination, of the features disclosed in this specification (including any accompanying claims, abstract, and drawings), or to any novel one, or any novel combination, of the steps of any method or process so disclosed.

As used in the specification and the appended claims, the singular forms “a”, “an”, and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about”, it will be understood that the particular value forms another aspect. It will be further understood that the endpoints of each of the ranges are significant both in relation to the other endpoint, and independently of the other endpoint. The terms “about” and “approximately” are defined as being “close to” as understood by one of ordinary skill in the art. In one non-limiting aspect the terms are defined to be within 10%. In another non-limiting aspect, the terms are defined to be within 5%. In still another non-limiting aspect, the terms are defined to be within 1%.

The terms “coupled”, “connected”, and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

Certain terminology is used in the following description for convenience only and is not limiting. The words “right”, “left”, “lower”, and “upper” designate direction in the drawings to which reference is made. The words “inner” and “outer” refer to directions toward and away from, respectively, the geometric center of the described feature or device. The words “distal” and “proximal” refer to directions taken in context of the item described and, with regard to the instruments herein described, are typically based on the perspective of the practitioner using such instrument, with “proximal” indicating a position closer to the practitioner and “distal” indicating a position further from the practitioner. The terminology includes the above-listed words, derivatives thereof, and words of similar import.

Throughout the description and claims of this specification, the word “comprise” and variations of the word, such as “comprising” and “comprises”, means “including but not limited to”, and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention.

What is claimed is:

1. A device for subdermal insertion of solid media, the device comprising:
a front body defining a window;
a rear body selectively coupled to the front body;
a needle hub received within the front body and the rear body and movable between an extended position and a retracted position; and
a trocar coupled to the needle hub and including a payload retention feature.

2. The device of claim 1, wherein the rear body includes an indicator window, and
wherein the needle hub includes a hub indicator tab that is viewable through the indicator window when the needle hub is arranged in the retracted position.

3. The device of claim 2, further comprising a plunger positioned within the rear body and including a plunger indicator tab viewable through the indicator window when the needle hub is arranged in the extended position.

4. The device of claim 3, wherein the plunger includes a rod sized to be received within the trocar and configured to eject a payload from the trocar.

5. The device of claim 1, wherein the needle hub includes a hub button configured to be engaged by a user to force the needle hub from the extended position to the retracted position.

6. The device of claim 1, wherein the trocar is a tab retainer trocar including a payload retainer tab.

7. The device of claim 1, wherein the trocar is a sleeve tip retainer trocar including a polymer sleeve.

8. The device of claim 1, wherein the trocar is a foil retainer trocar including a flat shaped foil or a v-shaped foil.

9. The device of claim 1, wherein the trocar is a sleeve slot retainer trocar that defines a slot and includes a polymer sleeve covering the slot.

10. The device of claim 1, wherein the trocar is a plug retainer trocar including a plug.

11. The device of claim 1, wherein the rear body includes a rod sized to be received within the trocar and configured to eject a payload from the trocar.

12. The device of claim 1, wherein the needle hub includes a hub button defining a pulling surface at a distal end of the hub button.

13. The device of claim 12, wherein the pulling surface intersects a proximal window plane intersecting a proximal end of the window.

14. The device of claim 1, wherein the window defines a window width of 14 mm or less.

15. The device of claim 1, wherein the window is defined by a hoop having a width wider than a hand grip.

16. The device of claim 1, wherein the rear body defines a swept tail configured to be pushed by a user.

17. The device of claim 1, wherein the front body defines a textured grip surface.

18. A device for subdermal insertion of solid media, the device comprising:
a body includes a hand grip and a loop defining a window, the loop defining a width wider than the hand grip;
a needle hub slidably received within the body movable between an extended position and a retracted position; and
a trocar coupled to the needle hub and including a payload retention feature.

19. The device of claim 18, wherein the trocar defines a cutting tip, and
wherein the cutting tip is visible through the window when the needle hub is arranged in the extended position.

20. A device for subdermal insertion of solid media, the device comprising:
a front body includes a hand grip and a loop defining a window and a proximal window plane intersecting a proximal end of the window, wherein the window defines a window width of 14 mm or less;
a rear body selectively coupled to the front body, the rear body defining a swept tail configured to be pushed by a user;
a needle hub received within the front body and the rear body and movable between an extended position and a retracted position, the needle hub including a hub button defining a pulling surface at a distal end of the hub button, wherein the pulling surface intersects the proximal window plane; and
a trocar coupled to the needle hub and including a payload retention feature.
